(19) United States
(12) Patent Application Publication
(10) Pub. No.: US 2023/0212257 A1
(43) Pub. Date: Jul. 6, 2023

(54) ANTI-CD171 CHIMERIC ANTIGEN RECEPTORS

(71) Applicant: Seattle Children's Hospital (dba Seattle Children's Research Institute), Seattle, WA (US)

(72) Inventors: Michael C. Jensen, Seattle, WA (US); Adam Johnson, Seattle, WA (US)

(21) Appl. No.: 18/000,468

(22) PCT Filed: Jun. 7, 2021
(86) PCT No.: PCT/US2021/036171

§ 371 Date: Dec. 1, 2022

Related U.S. Application Data
(60) Provisional application No. 63/036,021, filed on Jun. 8, 2020.

(51) Int. Cl. C07K 14/725; C07K 16/28

(52) U.S. Cl. CPC C07K 14/7051; C07K 16/2803; C07K 2319/03; C07K 2319/02

(57) ABSTRACT

Embodiments of the methods and compositions provided herein relate to anti-CD171 chimeric antigen receptors (CARs). Some embodiments relate to anti-CD171 CARs having long extracellular polypeptide spacers. Some embodiments relate to cells containing such anti-CD171 CARs having increased persistence and activity at a lower dose in a subject compared to cells containing anti-CD171 CARs comprising shorter polypeptide spacers.

### CROSS-REFERENCE TO RELATED APPLICATIONS

This application claims priority to U.S. Prov. App. No. 63/036,021 filed Jun. 8, 2020 entitled “ANTI-CD171 CHIMERIC ANTIGEN RECEPTORS” which is hereby expressly incorporated by reference herein in its entirety.

### REFERENCE TO SEQUENCE LISTING

The present application is being filed along with a Sequence Listing in electronic format. The Sequence Listing is provided as a file entitled SCRI283WOSEQLIST.TXT, created Jun. 4, 2021, which is approximately 16 Kb in size. The information in the electronic format of the Sequence Listing is incorporated herein by reference in its entirety.

### FIELD OF THE INVENTION

Embodiments of the methods and compositions provided herein relate to anti-CD171 chimeric antigen receptors (CARs). Some embodiments relate to anti-CD171 CARs having long extracellular polypeptide spacers. Some embodiments relate to cells containing such anti-CD171 CARs having increased persistence and activity at a lower dose in a subject compared to cells containing anti-CD171 CARs comprising shorter polypeptide spacers.

### BACKGROUND OF THE INVENTION

Neuroblastoma is the most common extracranial solid tumor of childhood with a heterogeneous clinical course. While neuroblastomas with favorable biology spontaneously regress or differentiate without therapeutic intervention, neuroblastomas with unfavorable biology often fatally progress despite intensive multimodal therapy. Maximally tolerated frontline intensive chemotherapy, radiation, consolidative autologous hematopoietic stem cell transplantation followed by retinoids and anti-GD2 antibody may cure up to 50% of high-risk patients. Accordingly, the development of new therapeutic modalities, which are tolerable in this patient population, is needed.

### SUMMARY OF THE INVENTION

Some embodiments of the methods and compositions provided herein include a nucleic acid comprising a polynucleotide encoding a chimeric antigen receptor (CAR), wherein the CAR comprises: a ligand binding domain capable of or configured to specifically bind to CD171; a polypeptide spacer having a length greater than or equal to 120 and less than or equal to 230 consecutive amino acid residues; a transmembrane domain; and an intracellular signalling domain.

In some embodiments, the ligand binding domain is derived from a CE7 monoclonal antibody.

In some embodiments, the ligand binding domain comprises an scFv comprising a nucleotide sequence having at least 95% sequence identity to the nucleotide sequence of SEQ ID NO:01.

In some embodiments, the polypeptide spacer comprises an IgG4 hinge-CH2-CH3 domain comprising an L235D substitution. In some embodiments, the polypeptide spacer comprises an amino acid sequence having at least 95% sequence identity to the amino acid sequence of SEQ ID NO:02.

In some embodiments, the transmembrane domain comprises a CD28 transmembrane domain. In some embodiments, the CD28 transmembrane domain comprises an amino acid sequence having at least 95% sequence identity to the amino acid sequence of SEQ ID NO:04.

In some embodiments, the intracellular signalling domain comprises a costimulatory domain selected from the group consisting of CD27, CD28, 4-1BB, OX-40, CD30, CD40, PD-1, ICOS, LFA-1, CD2, CD7, NKG2C, and B7-H3, in combination with a CD3-zeta domain or functional portion thereof. In some embodiments, the intracellular signalling domain comprises a CD28 cytoplasmic domain. In some embodiments, the CD28 cytoplasmic domain comprises an amino acid sequence having at least 95% sequence identity to the amino acid sequence of SEQ ID NO:18. In some embodiments, the intracellular signalling domain lacks a CD28 cytoplasmic domain. In some embodiments, the intracellular signalling domain comprises a 4-1BB costimulatory domain. In some embodiments, the 4-1BB costimulatory domain is encoded by a nucleotide sequence having at least 95% sequence identity to the nucleotide sequence of SEQ ID NO:07. In some embodiments, the CD3-zeta domain or functional portion thereof is encoded by a nucleotide sequence having at least 95% sequence identity to the nucleotide sequence of SEQ ID NO:08.

Some embodiments also include a constitutive promoter operably linked to the polynucleotide encoding a chimeric antigen receptor. In some embodiments, the constitutive promoter comprises an EF1a promoter.

Some embodiments also include an inducible promoter operably linked to the polynucleotide encoding a chimeric antigen receptor.

Some embodiments also include a polynucleotide encoding a cell-surface selectable marker. In some embodiments, the cell-surface selectable marker is selected from a truncated EGFR polypeptide (EGFRt) or a truncated Her2 polypeptide (Her2t).

Some embodiments also include a ribosome skip sequence located between a polynucleotide encoding the intracellular signalling domain and the polynucleotide encoding a cell-surface selectable marker. In some embodiments, the ribosome skip sequence is selected from the group consisting of a P2A sequence, a T2A sequence, an E2A sequence, and an F2A sequence.

Some embodiments also include a polynucleotide encoding a suicide gene system. In some embodiments, the suicide gene system is selected from a herpes simplex virus thymidine kinase/ganciclovir (HSVTK/GCV) suicide gene system, or an inducible caspase suicide gene system.

In some embodiments, the ligand binding domain comprises the nucleotide sequence of SEQ ID NO:01; the polypeptide spacer consists of the amino acid sequence of SEQ ID NO:02; the transmembrane domain comprises the amino acid sequence of SEQ ID NO:04; the intracellular signalling domain comprises: a 4-1BB costimulatory domain encoded by the nucleotide sequence of SEQ ID NO:07, and a CD3-zeta domain or functional portion thereof encoded by the nucleotide sequence of SEQ ID NO:08; and further comprising: an EF1 promoter operably linked to a polynucleotide encoding the ligand binding domain, a polynucleotide comprising a T2A ribosome skip sequence, and a polynucleotide encoding an EGFRt polypeptide.

Some embodiments of the methods and compositions provided herein include a polypeptide encoded by any one of the foregoing nucleic acids.

Some embodiments of the methods and compositions provided herein include a vector comprising any one of the foregoing nucleic acids. In some embodiments, the vector is selected from the group consisting of a viral vector, a transposon vector, an integrase vector, and an mRNA vector. In some embodiments, the viral vector is selected from the group consisting of a lentiviral vector, a foamy viral vector, a retroviral vector, and a gamma retroviral vector. In some embodiments, the viral vector is a lentiviral vector.

Some embodiments of the methods and compositions provided herein include a host cell comprising any one of the foregoing nucleic acids.

In some embodiments, the host cell is a CD4+ T-cell or a CD8+ T-cell.

In some embodiments, the host cell is a precursor T-cell, or a hematopoietic stem cell.

In some embodiments, the host cell is a CD8+ cytotoxic T-cell selected from the group consisting of a naïve CD8+ T-cell, a CD8+ memory T-cell, a central memory CD8+ T-cell, a regulatory CD8+ T-cell, an IPS derived CD8+ T-cell, an effector memory CD8+ T-cell, and a bulk CD8+ T-cell.

In some embodiments, the host cell is a CD4+ T helper cell selected from the group consisting of a naïve CD4+ T-cell, a CD4+ memory T-cell, a central memory CD4+ T-cell, a regulatory CD4+ T-cell, an IPS derived CD4+ T-cell, an effector memory CD4+ T-cell, and a bulk CD4+ T-cell.

In some embodiments, the cell is allogenic to a subject, preferably a human or autologous to a subject, preferably a human.

Some embodiments of the methods and compositions provided herein include a pharmaceutical composition comprising any one of the foregoing host cells and a pharmaceutically acceptable excipient.

Some embodiments of the methods and compositions provided herein include a method of treating, inhibiting or ameliorating a cancer in a subject, comprising administering any one of the foregoing host cells to the subject.

In some embodiments, the subject is administered a dose comprising a plurality of the host cell sufficient to treat, inhibit or ameliorate the cancer that is less than a dose sufficient to treat, inhibit or ameliorate the cancer of a plurality of cells comprising a CAR comprising a polypeptide spacer having a length less than 120 consecutive amino acid residues.

In some embodiments, a dose comprising a plurality of the host cell less than 5×10⁶ cell/kg is administered to the subject.

Some embodiments also include administering cetuximab to the subject.

In some embodiments, the cancer comprises a CD171 expressing cell. In some embodiments, the cancer is selected from a neuroblastoma or a ganglioneuroblastoma.

Some embodiments of the methods and compositions provided herein include any one of the foregoing host cells for use in treating, inhibiting or ameliorating a cancer in a subject.

Some embodiments of the methods and compositions provided herein include use of any one of the foregoing host cells in the manufacture of a medicament for treating, inhibiting or ameliorating a cancer in a subject.

Some embodiments of the methods and compositions provided herein include any one of the foregoing host cells for use as a medicament.

### BRIEF DESCRIPTION OF THE DRAWINGS

FIG. 1 depicts a schematic of an example structure of a nucleic acid encoding a second generation and a third generation anti-CD171 CAR with a short spacer. Encoded elements include an EF1 promoter, a leader sequence encoding a signal polypeptide, VH-linker-VL domains encoding an anti-CD171 scFv ligand binding domain, an IgG4-hinge spacer (short spacer), a CD28 transmembrane (CD28tm) domain, a 4-1BB domain, a CD3zeta domain, a T2A ribosome skip sequence, a truncated EGFR (tEGFR) polypeptide, which can be a cell surface selectable marker. Anti-CD171 CARs with long spacers include an IgG4 hinge-CH2-CH3 domain instead of an IgG4 hinge domain.

FIG. 2 depicts a series of line graphs of percentage specific lysis for various ratios of effector cells comprising anti-CD171 CARs co-cultured with CD171+ target cells or control target cells. CARs included: a second generation anti-CD171 CAR with short spacer (CE7 2G short); a third generation anti-CD171 CAR with short spacer (CE7 3G short); a second generation anti-CD171 CAR with long spacer (CE7 2G long); and a third generation anti-CD171 CAR with long spacer (CE7 3G long).

FIG. 3 depicts a series of graphs of cytokine production (IL-2, IFN-gamma, and TNF-alpha) for various effector CD4+ T cells comprising anti-CD171 CARs co-cultured with CD171+ target cells or control target cells. CARs included: a second generation anti-CD171 CAR with short spacer (CE7 2GS); a third generation anti-CD171 CAR with short spacer (CE7 3GS); a second generation anti-CD171 CAR with long spacer (CE7 2GL); and a third generation anti-CD171 CAR with long spacer (CE7 3GL).

FIG. 4 depicts a line graph for signal (flux) from in vivo tumor cells over time in an intracranial neuroblastoma xenograft model treated with effector cells comprising CARs. CARs included: a second generation anti-CD171 CAR with short spacer (CE7 2GS); a third generation anti-CD171 CAR with short spacer (CE7 3GS); a second generation anti-CD171 CAR with long spacer having two substitutions in the spacer (CE7 2GL, 2 mut); and a third generation anti-CD171 CAR with long spacer having two substitutions in the spacer (CE7 3GL, 2 mut).

FIG. 5 depicts a line graph for percent survival in a xenograft neuroblastoma model treated with effector cells comprising CARs. CARs included: a second generation anti-CD171 CAR with short spacer (CE7 2GS); a third generation anti-CD171 CAR with short spacer (CE7 3GS); a second generation anti-CD171 CAR with long spacer having two substitutions in the spacer (CE7 2GL, 2 mut); and a third generation anti-CD171 CAR with long spacer having two substitutions in the spacer (CE7 3GL, 2 mut).

### DETAILED DESCRIPTION

Embodiments of the methods and compositions provided herein relate to anti-CD171 chimeric antigen receptors (CARs). Some embodiments relate to anti-CD171 CARs having long extracellular polypeptide spacers. Some embodiments relate to cells containing such anti-CD171 CARs having increased persistence and activity at a lower dose in a subject compared to cells containing anti-CD171 CARs comprising shorter polypeptide spacers.

Patients with recurrent or refractory neuroblastoma are resistant to conventional chemotherapy. For this reason, investigators are attempting to use T cells obtained directly from the patient, which can be genetically modified to express a CAR. The CAR enables the T cell to recognize and kill the neuroblastoma cell through the recognition of CD171, a protein expressed of the surface of the neuroblastoma cell in patients with neuroblastoma.

Immunotherapy is an attractive approach because it invokes immunologic effector mechanisms to which chemotherapy/radiation-resistant tumor cells are susceptible. T cells expressing a CAR engage tumor cells independent of expression of HLA molecules and are activated via coordinated co-stimulation and CD3zeta signaling. A monoclonal antibody designated CE7 binds to an epitope on human L1CAM (CD171) in the context of tumor expression, a CE7 scFv was derived and assembled into a CAR for T cell-redirected tumor targeting (Hoefnagel C A., et al., (2001) Eur J Nucl Med 28:359-68). While the molecular basis responsible for the tumor selective CE7 epitope on L1CAM has not been fully elucidated, several lines of evidence suggest that binding is dependent on glycosylation. CD171 plays a role in oncogenesis as its expression correlates with tumor progression and metastasis in several solid tumors and participates in the regulation of tumor cell differentiation, proliferation, migration, and invasion. Initial assessment of target safety was made in a previously reported pilot clinical trial using autologous cloned CD8+ cytolytic T lymphocytes expressing a first-generation CE7-CAR (Park J. et al., (2007) Mol Ther 15:825-33). Six neuroblastoma patients were treated with doses up to 10⁹ cells/m² without obvious off-tumor toxicity. However, the duration and magnitude of persistence were limited. CE7-CARs have been generated containing a short spacer extracellular domain and one (4-1BB; second-generation CAR) or two (CD28 and 4-1BB; third generation CAR) intracellular costimulatory signaling domains. A phase I clinical trial to determine the safety, feasibility, and optimal dose of 2G and 3G CE7-CAR T cells in patients with refractory or relapsed neuroblastoma has been initiated (ClinicalTrials.gov Identifier: NCT02311621).

Certain aspects useful with embodiments of the methods and compositions provided herein are disclosed in U.S. 2018/0009891, U.S. 2017/0267742, U.S. 2017/0015746, Kunkele A. et al., (2015) Cancer Immunol Res 3:368-379, and Kunkele A., et al., (2017) Clin Cancer Res 23:466-477, which are each expressly incorporated by reference in its entirety.

### Definitions

Unless defined otherwise, all technical and scientific terms used herein have the same meaning as commonly understood by one of ordinary skill in the art to which the invention pertains.

As used herein, “a” or “an” may mean one or more than one.

“About” as used herein when referring to a measurable value is meant to encompass variations of ±20% or ±110%, more preferably ±5%, even more preferably ±1%, and still more preferably ±0.1% from the specified value.

As used herein, “nucleic acid” or “nucleic acid molecule” have their plain and ordinary meaning in view of the whole specification and may to refer to, for example, polynucleotides, such as deoxyribonucleic acid (DNA) or ribonucleic acid (RNA), oligonucleotides, fragments generated by the polymerase chain reaction (PCR), or fragments generated by any of ligation, scission, endonuclease action, or exonuclease action. Nucleic acid molecules can be composed of monomers that are naturally occurring nucleotides (such as DNA or RNA), or analogs of naturally occurring nucleotides (e.g., enantiomeric forms of naturally occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, or azido groups, or sugars can be functionalized as ethers or esters. Moreover, the entire sugar moiety can be replaced with sterically and electronically similar structures, such as aza-sugars or carbocyclic sugar analogs. Examples of modifications in a base moiety include alkylated purines or pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate, and the like. The term “nucleic acid molecule” also includes so-called “peptide nucleic acids,” which comprise naturally occurring or modified nucleic acid bases attached to a polyamide backbone. Nucleic acids can be either single stranded or double stranded. In some embodiments, a nucleic acid sequence encoding a fusion protein is provided. In some embodiments, the nucleic acid encoding the CAR specific for CD171 is RNA or DNA.

As used herein, “coding for” or “encoding” has its plain and ordinary meaning when read in light of the specification, and includes, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

As used herein, “chimeric antigen receptor” (CAR) has its plain and ordinary meaning when read in light of the specification, and may include but is not limited to, for example, a synthetically designed receptor comprising a ligand binding domain of an antibody or other protein sequence that binds to a molecule associated with a disease or disorder and is, preferably, linked via a spacer domain to one or more intracellular signaling domains of a cell, such as a T cell, or other receptors, such as one or more costimulatory domains. Chimeric receptor can also be referred to as artificial cell receptors or T cell receptors, chimeric cell receptors or T cell receptors, chimeric immunoreceptors, or CARs. These receptors can be used to graft the specificity of a monoclonal antibody or binding fragment thereof onto a cell, preferably a T-cell, with transfer of their coding sequence facilitated by viral vectors, such as a retroviral vector or a lentiviral vector. CARs can be, in some instances, genetically engineered T cell receptors designed to redirect T cells to target cells that express specific cell-surface antigens. T cells can be removed from a subject and modified so that they can express receptors that can be specific for an antigen by a process called adoptive cell transfer. The T cells are reintroduced into the patient where they can then recognize and target an antigen. CARs are also engineered receptors that can graft an arbitrary specificity onto an immune receptor cell. CARs are considered by some investigators to include the antibody or antibody fragment, preferably an antigen binding fragment of an antibody, the spacer, signaling domain, and transmembrane region. Due to the surprising effects of modifying the different components or domains of the CAR described herein, such as the epitope binding region (for example, antibody fragment, scFv, or portion thereof), spacer, transmembrane domain, and/or signaling domain), the components of the CAR are frequently distinguished throughout this disclosure in terms of independent elements. The variation of the different elements of the CAR can, for example, lead to a desired binding affinity, such as a stronger binding affinity for a specific epitope or antigen.

The CARs graft the specificity of a monoclonal antibody or binding fragment thereof or scFv onto a T cell, with the transfer of their coding sequence facilitated by vectors. In order to use CARs as a therapy for a subject in need, a technique called adoptive cell transfer is used in which T cells are removed from a subject and modified so that they can express the CARs that are specific for an antigen. The T cells, which can then recognize and target an antigen, are reintroduced into the patient.

In some embodiments, the transmembrane domain is a region of a membrane-spanning protein that is hydrophobic that can reside in the bilayer of a cell to anchor a protein that is embedded to the biological membrane. Without being limiting, the topology of the transmembrane domain can be a transmembrane alpha helix. In some alternatives of the chimeric antigen receptor, the CAR comprises a sequence encoding a transmembrane domain. In some alternatives, the transmembrane domain comprises a CD28 transmembrane sequence or a fragment thereof that is a length of 10, 11, 12, 13, 14, 15, 16, 17, 18, 19, 20, 21, 22, 23, 24, 25, 26, 27, or 28 amino acids or a length within a range defined by any two of the aforementioned lengths. In some alternatives, the CD28 transmembrane sequence or fragment thereof comprises 28 amino acids in length.

In some embodiments, the signaling domains, such as primary signaling domains or costimulatory domains, include an intracellular or cytoplasmic domain of a protein or a receptor protein that interacts with components within the interior of the cells and is capable of or configured to relay or participate in the relaying of a signal. Such interactions in some aspects can occur through the intracellular domain communicating via specific protein-protein or protein-ligand interactions with an effector molecule or an effector protein, which in turn can send the signal along a signal chain to its destination. In some embodiments, the signaling domain includes one or more co-stimulatory domains. In some aspects, the one or more costimulatory domains include a signaling moiety that provides a T-cell with a signal, which, in addition to the primary signal provided by for instance the CD3 zeta chain of the TCR/CD3 complex, enhances a response such as a T-cell effector response, such as, for example, an immune response, activation, proliferation, differentiation, cytokine secretion, cytolytic activity, perforin or granzyme activity or any combination thereof. In some embodiments, the intracellular signaling domain or the co-stimulatory domain can include all or a portion of CD27, CD28, 4-1BB, OX40, CD30, CD40, ICOS, lymphocyte function-associated antigen-1 (LFA-1), CD2, CD7, LIGHT, NKG2C, or B7-H3, or a ligand that specifically binds with CD83 or any combination thereof.

As used herein, an “antibody” has its plain and ordinary meaning when read in light of the specification, and includes, for example, a large Y-shape protein produced by plasma cells that is used by the immune system to identify and neutralize foreign objects such as bacteria and viruses. The antibody protein can comprise four polypeptide chains, two identical heavy chains and two identical light chains connected by disulfide bonds. Each chain is composed of structural domains called immunoglobulin domains. These domains can contain about 70, 80, 90, 100, 110, 120, 130, 140, or 150 amino acids or any number of amino acids in between in a range defined by any two of these values and are classified into different categories according to their size and function. In some embodiments, the ligand binding domain comprises an antibody or binding fragment thereof or scFv, a receptor ligand or mutants thereof, peptide, and/or polypeptide affinity molecule or binding partner. In some embodiments, the ligand binding domain is an antibody fragment, desirably, a binding portion thereof. In some embodiments, the antibody fragment or binding portion thereof present on a CAR is specific for a ligand on a B-cell. In some embodiments, the antibody fragment or binding portion thereof present on a CAR or TcR is specific for a ligand. In some embodiments, the antibody fragment or binding portion thereof present on a CAR is specific for CD171. In some embodiments, the ligand binding domain is an antibody fragment or a binding portion thereof, such as a single chain variable fragment (scFv). In some embodiments, the antibody fragment or binding portion thereof present on a CAR comprises one or more domains from a humanized antibody, or binding portion thereof.

As used herein, a “single chain variable fragment” or “scFv” has its plain and ordinary meaning when read in light of the specification, and includes, for example, a fusion protein of the variable regions of the heavy (VH) and light chains (VL) of immunoglobulins, connected with a short linker peptide of ten or about ten amino acids to 25 or about 25 amino acids. In some embodiments, a CAR is provided, wherein the CAR comprises a ScFv specific for CD171.

The strength of binding of a ligand is referred to as the binding affinity and can be determined by direct interactions and solvent effects. A ligand can be bound by a “ligand binding domain.” A ligand binding domain, for example, can refer to a conserved sequence in a structure that can bind a specific ligand or a specific epitope on a protein. The ligand binding domain or ligand binding portion can comprise an antibody or binding fragment thereof or scFv, a receptor ligand or mutants thereof, peptide, and/or polypeptide affinity molecule or binding partner. Without being limiting, a ligand binding domain can be a specific protein domain or an epitope on a protein that is specific for a ligand or ligands.

Some embodiments include a spacer. In some alternatives, the peptide spacer is 15 amino acids or less but not less than 1 or 2 amino acids. In some embodiments, the spacer is a polypeptide chain. In some aspects, the polypeptide chain may range in length, such as from 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217,218, 219, 220, 221,222, 223, 224, 225, 226, 227, 228, 229, 230, 231, 232, 233, 234, 235, 236, 237, 238, 239 or 240 consecutive amino acids or have a length within a range defined by any two of the aforementioned lengths. A spacer can comprise any 20 amino acids, for example, in any order to create a desirable length of polypeptide chain in a chimeric antigen receptor, preferably the amino acids arginine, histidine, lysine, aspartic acid, glutamic acid, serine, threonine, asparagine, glutamine, cysteine, glycine, proline, alanine, valine, isoleucine, methionine, phenylalanine, tyrosine or tryptophan. A spacer sequence can be a linker between the scFv (or ligand binding domain) and the transmembrane domain of the chimeric antigen receptor. In some alternatives, the CAR further comprises a sequence encoding a spacer. In some alternatives, the spacer comprises a sequence with a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122,123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239 or 240 amino acids or a length within a range defined by any two of the aforementioned lengths. In some alternatives, the spacer resides between the scFv and the transmembrane region of the CAR. In some alternatives, the spacer resides between the ligand binding domain of the CAR and the transmembrane region of the CAR.

A spacer may also be customized, selected, or optimized for a desired length so as to improve or modulate binding of scFv domain to the target cell, which may increase or provide the desired amount of cytotoxic efficacy. In some alternatives, the linker or spacer between the scFv domain or ligand binding domain and the transmembrane can be 25 to 55 amino acids in length (e.g., at least, equal to 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids or a length within a range defined by any two of the aforementioned lengths).

In some alternatives, the spacer comprises a hinge region of a human antibody. In some alternatives, the spacer comprises an IgG4 hinge. In some alternatives, the IgG4 hinge region is a modified IgG4 hinge.

As used herein, a “de-immunized spacer” has its plain and ordinary meaning when read in light of the specification, and includes, for example, a spacer that induces little to no immune response or a diminished or reduced immune response from a patient. In some embodiments, the CAR comprises a spacer, wherein the spacer does not induce an immune response in a subject, such as a human. It is important that the spacer does not induce an immune response or induces a reduced or diminished or low immune response in a subject, such as a human, in order to prevent or reduce the ability of the immune system to attack the CAR

As used herein, a “ribosome skip sequence” has its plain and ordinary meaning when read in light of the specification, and includes, for example, a sequence that during translation, forces the ribosome to “skip” the ribosome skip sequence and translate the region after the ribosome skip sequence without formation of a peptide bond. Several viruses, for example, have ribosome skip sequences that allow sequential translation of several proteins on a single nucleic acid without having the proteins linked via a peptide bond. As described herein, this is the “linker” sequence. In some alternatives of the nucleic acids provided herein, the nucleic acids comprise a ribosome skip sequence between the sequence for the chimeric antigen receptor and the sequence of the marker protein, such that the proteins are co-expressed and not linked by a peptide bond. In some embodiments, the ribosome skip sequence is a P2A, T2A, E2A or F2A sequence.

As used herein, a “marker sequence,” has its plain and ordinary meaning when read in light of the specification, and includes, for example, a protein that is used for selecting or tracking a protein or cell that has a protein of interest. In the alternatives described herein, the fusion protein provided can comprise a marker sequence that can be selected in experiments, such as flow cytometry. In some embodiments, the marker comprises a truncated Her2 (Her2t) polypeptide, or a truncated EGFR (EGFRt).

As used herein, “signal sequence” for secretion, can also be referred to as a “signal peptide.” The signal peptide can be used for secretion efficiency and in some systems, it is recognized by a signal recognition particle, which halts translation and directs the signal sequence to an SRP receptor for secretion. In some alternatives of the CARs provided herein, the CARs further comprise a signal sequence. In some embodiments, of the nucleic acid encoding a CAR, the nucleic acid comprises a sequence encoding a signal sequence. In some embodiments, the signal sequence is for targeting a protein to a cell membrane following translation of the protein.

As used herein, “suicide gene therapy,” “suicide genes” and “suicide gene systems” have their plain and ordinary meaning when read in light of the specification, and includes, for example, methods to destroy a cell through apoptosis, which requires a suicide gene that will cause a cell to kill itself by apoptosis. Due to safety concerns for the patients in need of using genetically modified immune cells for treatment or modification of a tumor environment, strategies are being developed in order to prevent or abate adverse events. Adverse effects of incorporation of genetically modified immune cells into a subject for a pretreatment step can include “cytokine storms,” which is a cytokine release syndrome, wherein the infused T-cells release cytokines into the bloodstream, which can lead to dangerously high fevers, as well as, a precipitous drop in blood pressure. Control of the system by tamoxifen, as previously described, may also be used when there is indication of a cytokine storm, such as a fever.

As used herein, “vector” or “construct” has its plain and ordinary meaning when read in light of the specification, and includes, for example, a nucleic acid used to introduce heterologous nucleic acids into a cell that has regulatory elements to provide expression of the heterologous nucleic acids in the cell. Vectors include but are not limited to plasmid, minicircles, yeast, viral genomes, lentiviral vector, foamy viral vector, retroviral vector or gammaretroviral vector. The vector may be DNA or RNA, such as mRNA.

As used herein, “transposon gene cassettes” or transposons refers to a genetic element that contains a gene (promoter that drives expression of a primary transcript), flanked by recombinase recognition sites (for example, Sleeping Beauty transposase recognition sites, or PiggyBac). The transposon gene cassette may be incorporated into an integrated genomic sequence or may exist freely as circular DNA. In some embodiments, the transposon gene cassette encodes a promoter, a CAR, and a signal sequence to direct the protein to the cell surface.

As used herein, “integrase vector systems” work by integrating a viral donor nucleic acid with specific attachment sites to a target genome. Through the use of integrase, the viral DNA is inserted into the host DNA.

As used herein, “T-cells” or “T lymphocytes” can be from any mammal, preferably a primate, including monkeys or humans, a companion animal such as a dog, cat, or horse, or a domestic animal, such as a sheep, goat, or cattle. In some alternatives the T-cells are allogeneic (from the same species but different donor) as the recipient subject; in some alternatives the T-cells are autologous (the donor and the recipient are the same); in some alternatives the T-cells are syngeneic (the donor and the recipients are different but are identical twins).

As used herein, “T cell precursors” refers to lymphoid precursor cells that can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8 and are therefore classed as double-negative (CD4−CD8−) cells. As they progress through their development, they become double-positive thymocytes (CD4+CD8+), and finally mature to single-positive (CD4+CD8− or CD4−CD8+) thymocytes that are then released from the thymus to peripheral tissues.

As used herein, “hematopoietic stem cells” or “HSC” are precursor cells that can give rise to myeloid cells such as, for example, macrophages, monocytes, macrophages, neutrophils, basophils, eosinophils, erythrocytes, megakaryocytes/platelets, dendritic cells and/or lymphoid lineages (such as, for example, T-cells, B-cells, or NK-cells). HSCs have a heterogeneous population in which three classes of stem cells exist, which are distinguished by their ratio of lymphoid to myeloid progeny in the blood (UJM).

As used herein, “CD4+ expressing T-cell,” or “CD4+ T-cell,” are used synonymously throughout, is also known as T helper cells, which play an important role in the immune system, and in the adaptive immune system. CD4+ T-cells also help the activity of other immune cells by releasing T-cell cytokines. These cells help, suppress or regulate immune responses. They are essential in B cell antibody class switching, in the activation and growth of cytotoxic T-cells, and in maximizing bactericidal activity of phagocytes, such as macrophages. CD4+ expressing T-cells have the ability to make some cytokines, however the amounts of cytokines made by CD4+ T-cells are not at a concentration that promotes, improves, contributes to, or induces engraftment fitness. As described herein, “CD4+ T-cells” are mature T helper-cells that play a role in the adaptive immune system.

As used herein, “CD8+ expressing T-cell” or “CD8+ T-cell,” are used synonymously throughout, is also known as a TC, cytotoxic T lymphocyte, CTL, T-killer cell, cytolytic T-cell or killer T-cell. As described herein, CD8+ T-cells are T-lymphocytes that can kill cancer cells, virally infected cells, or damaged cells. CD8+ T-cells express T-cell receptors (TCRs) that can recognize a specific antigen. CD8+ T-cells express CD8 on the surface. CD8+ expressing T-cells have the ability to make some cytokines, however the amounts of cytokines made by CD8+ T-cells are not at a concentration that promotes, improves, contributes to, or induces engraftment fitness. “CD8 T-cells” or “killer T-cells” are T-lymphocytes that can kill cancer cells, cells that are infected with viruses or cells that are damaged.

Mature T cells express the surface protein CD4 and are referred to as CD4+ T-cells. CD4+ T-cells are generally treated as having a pre-defined role as helper T-cells within the immune system. For example, when an antigen-presenting cell expresses an antigen on MHC class II, a CD4+ cell will aid those cells through a combination of cell to cell interactions (e.g. CD40 and CD40L) and through cytokines. Nevertheless, there are rare exceptions; for example, sub-groups of regulatory T-cells, natural killer cells, and cytotoxic T-cells express CD4. All of the latter CD4+ expressing T-cell groups are not considered T helper cells.

As used herein, “central memory” T-cell (or “TCM”) refers to an antigen experienced CTL that expresses CD62L or CCR-7 and CD45RO on the surface thereof and does not express or has decreased expression of CD45RA as compared to naïve cells. In some embodiments, central memory cells are positive for expression of CD62L, CCR7, CD28, CD127, CD45RO, and/or CD95, and have decreased expression of CD54RA, as compared to naïve cells.

As used herein, “effector memory” T-cell (or “TEM”) refers to an antigen experienced T-cell that does not express or has decreased expression of CD62L on the surface thereof as compared to central memory cells, and does not express or has decreased expression of CD45RA as compared to naïve cell. In some embodiments, effector memory cells are negative for expression of CD62L and/or CCR7, as compared to naïve cells or central memory cells, and have variable expression of CD28 and/or CD45RA.

As used herein, “naïve” T-cells refers to a non-antigen experienced T lymphocyte that expresses CD62L and/or CD45RA, and/or does not express CD45RO− as compared to central or effector memory cells. In some embodiments, naïve CD8+ T lymphocytes are characterized by the expression of phenotypic markers of naïve T-cells including CD62L, CCR7, CD28, CD127, or CD45RA.

As used herein, “effector” “TE” T-cells refers to a antigen experienced cytotoxic T lymphocyte cells that do not express or have decreased expression of CD62L, CCR7, CD28, and are positive for granzyme B or perforin or both, as compared to central memory or naïve T-cells.

As used herein, “engraftment fitness” has its plain and ordinary meaning when read in light of the specification, and includes, for example, the ability of a cell to grow and proliferate after the cells have entered the body, e.g., blood stream, through adoptive transfer. Engraftment can usually occur within two to four weeks after the transfer. Engraftment can be monitored by checking blood counts for a specific cell on a frequent basis. In some alternatives of the method of treating, inhibiting, or ameliorating a disease in a subject is provided, the method can comprise administering a composition or product combination comprising the genetically modified T-cells, as described herein. In some embodiments, the method can further comprise monitoring the subject by checking the blood counts for the genetically modified T-cells that expresses a chimeric antigen receptor e.g., by identifying the presence or absence of a marker associated with the transferred T-cells.

As used herein, “protein” has its plain and ordinary meaning when read in light of the specification, and includes, for example, a macromolecule comprising one or more polypeptide chains. A protein can therefore comprise of peptides, which are chains of amino acid monomers linked by peptide (amide) bonds, formed by any one or more of the amino acids. A protein or peptide can contain at least two amino acids, and no limitation is placed on the maximum number of amino acids that can comprise the protein or peptide sequence. Without being limiting, the amino acids are, for example, arginine, histidine, lysine, aspartic acid, glutamic acid, serine, threonine, asparagine, glutamine, cysteine, cystine, glycine, proline, alanine, valine, hydroxyproline, isoleucine, leucine, pyrolysine, methionine, phenylalanine, tyrosine, tryptophan, ornithine, S-adenosylmethionine, or selenocysteine. A protein can also comprise non-peptide components, such as carbohydrate groups, for example. Carbohydrates and other non-peptide substituents can be added to a protein by the cell in which the protein is produced and will vary with the type of cell. Proteins are defined herein in terms of their amino acid backbone structures; substituents such as carbohydrate groups are generally not specified, but can be present, nonetheless. In some embodiments, a CAR T-cell is also engineered to further express a protein, such as a cytokine, a chimeric cytokine receptor, a chimeric costimulatory molecule, a dominant negative receptor, an immunostimulatory molecule, or an immunoregulatory molecule.

As used herein, “cytokines” has its plain and ordinary meaning when read in light of the specification, and includes, for example, small proteins (5-25 kDa) that are important in cell signaling. Cytokines are released by cells and affect the behavior of other cells, and sometimes the releasing cell itself, such as a T-cell. Cytokines can include, for example, chemokines, interferons, interleukins, lymphokines, or tumor necrosis factor or any combination thereof. Cytokines can be produced by a broad range of cells, which can include, for example, immune cells like macrophages, B lymphocytes, T lymphocytes and mast cells, as well as, endothelial cells, fibroblasts, and various stromal cells.

As used herein, “interleukins” or IL are cytokines that the immune system depends largely upon. Examples of interleukins, which can be utilized herein, for example, include IL-1, IL-2, IL-3, IL-4, IL-5, IL-6, 11-7, IL-8/CXCL8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, or IL-36 or any combination thereof. Contacting T-cells with interleukins can have effects that promote, support, induce, or improve engraftment fitness of the cells. IL-1, for example can function in the maturation & proliferation of T-cells. IL-2, for example, can stimulate growth and differentiation of T-cell response. IL-3, for example, can promote differentiation and proliferation of myeloid progenitor cells. IL-4, for example, can promote proliferation and differentiation. IL-7, for example, can promote differentiation and proliferation of lymphoid progenitor cells, involved in B, T, and NK cell survival, development, and homeostasis. IL-15, for example, can induce production of natural killer cells. IL-21, for example, co-stimulates activation and proliferation of CD8+ T-cells, augments NK cytotoxicity, augments CD40-driven B cell proliferation, differentiation and isotype switching, and promotes differentiation of Th17 cells.

As used herein, “propagating cells” or propagation refers to steps to allow proliferation, expansion, growth and reproduction of cells. For example, cultures of CD8+ T-cells and CD4+ T-cells can typically be incubated under conditions that are suitable for the growth and proliferation of T lymphocytes. In some alternatives of the method of making genetically modified T-cells, which have a chimeric antigen receptor, the CD4+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods. In some alternatives of the method of making genetically modified T-cells, which have a chimeric antigen receptor, the CD8+ expressing T-cells are propagated for at least 1 day and may be propagated for 20 days, such as 1, 2, 3, 4, 5, 6, 7, 8, 9, 10, 11, 12, 13, 14, 15, 16, 17, 18, 19, or 20 days or for a period that is within a range defined by any two of the aforementioned time periods.

As used herein, “affinity selection,” refers to the selection of a specific molecule or cell having a selectable cell surface marker by binding to the molecule or marker or an epitope present thereon with a binding affinity agent, which allows for one to select out the specific molecule or cell of interest. Affinity selection can be performed by, for example, antibodies, conjugated antibodies, lectins, aptamers, or peptides or any combination thereof. In some embodiments, of the method of making genetically modified T-cells, the separating of the CD8+ population of T-cells and/or a CD4+ population of T-cells from a mixed population of T-cells is performed by affinity selection for T-cells having an epitope present on CD8 and/or CD4. In some alternatives of the method, anti-CD8 or anti-CD4 antibodies or binding portions thereof are used to select out the cells of interest. In some alternatives of the method, the separating of the CD8+ population of T-cells and/or a CD4+ population of T-cells from a mixed population of T-cells is performed by flow cytometry. In some alternatives of the method, the separating of the CD8+ population of T-cells and/or a CD4+ population of T-cells from a mixed population of T-cells is performed by immuno-magnetic selection. In some alternatives of the methods, the anti-CD8 or the anti-CD4 antibodies or binding fragments thereof are conjugated to a solid support such as, for example, an inert bead or an inert particle.

In another alternative, the expansion method or propagation can further comprise adding anti-CD3 and/or anti CD28 antibody to the culture medium (e.g., at a concentration of at least 0.5 ng/ml). In another alternative, the method of making genetically modified T-cells, which have a chimeric antigen receptor method can further comprise adding IL-2, IL-15, or IL-21 or any combination thereof to the culture medium (e.g., wherein the concentration of IL-2 is at least 10 units/ml). In another alternative, the method of making genetically modified T-cells, which have a chimeric antigen receptor method can further comprise adding IL-7, IL-I5, or IL-21 or any combination thereof to the culture medium (e.g., wherein the concentration of IL-2 is at least 10 units/ml). After isolation of T lymphocytes, both cytotoxic and helper T lymphocytes can be sorted into naïve, memory, and effector T-cell subpopulations either before or after expansion.

Some embodiments include polypeptide sequences or conservative variations thereof, such as conservative substitutions in a polypeptide sequence. In some embodiments, “conservative amino acid substitution” refers to amino acid substitutions that substitute functionally equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several families of conservative amino acid substitutions are shown in TABLE 1.

TABLE 1

| Family | Amino Acids |
|---|---|
| non-polar | Trp, Phe, Met, Leu, Ile, Val, Ala, Pro |
| uncharged polar | Gly, Ser, Thr, Asn, Gln, Tyr, Cys |
| acidic/negatively charged | Asp, Glu |
| basic/positively charged | Arg, Lys, His |
| Beta-branched | Thr, Val, Ile |
| residues that influence | Gly, Pro |
| chain orientation | |
| aromatic | Trp, Tyr, Phe, His |

### Certain Nucleic Acids

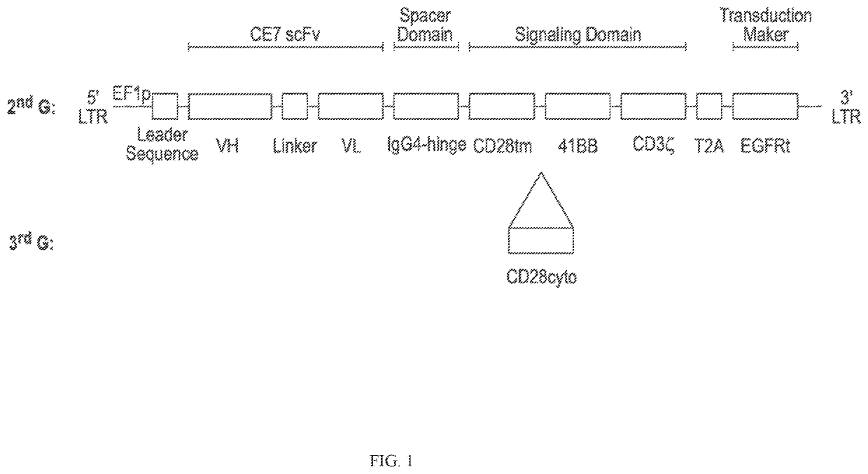

Some alternatives of the methods and compositions provided herein include a nucleic acid comprising a polynucleotide encoding a chimeric antigen receptor (CAR). TABLE 2 lists sequences useful with certain embodiments provided herein. FIG. 1 depicts exemplary embodiments of nucleic acids encoding a CAR. In some embodiments, the CAR comprises a ligand binding domain capable of or configured to specifically bind to CD171. In some embodiments, the ligand binding domain comprises a complementarity-determining region (CDR) derived from an antibody that specifically binds to CD171. In some embodiments, the ligand binding domain comprises a VH domain and/or a VL domain derived from an antibody that specifically binds to CD171. In some embodiments, the ligand binding domain is derived from a CE7 monoclonal antibody (see e.g., Schonmann S M, et al., (1986) Int J Cancer 1986; 37.255-62 which is expressly incorporated by reference in its entirety). In some embodiments, the ligand binding domain comprises an scFv encoded by a nucleotide sequence having a percentage sequence identity to the nucleotide sequence of SEQ ID NO:01 of at least 90%, 91%, 92%, 93%, 94%, 95%, 96%, 97%, 98%, or 99% or within a range defined by any two of the aforementioned percentages. In some embodiments, the ligand binding domain comprises an scFv comprising or consisting of a polypeptide encoded by the nucleotide sequence of SEQ ID NO:01.

In some embodiments, the CAR comprises a polypeptide spacer. In some embodiments, the polypeptide spacer has a length greater than or equal to 120 and less than or equal to 230 consecutive amino acid residues. In some embodiments, the polypeptide spacer consists of 229 consecutive amino acid residues. In some embodiments, the polypeptide spacer comprises an IgG4 hinge domain. In some embodiments, the polypeptide spacer comprises an IgG4 hinge-CH2-CH3 domain. In some embodiments, the polypeptide spacer comprises an IgG4 hinge-CH2-CH3 domain in which the CH2 domain includes an L235D substitution. In some embodiments, the polypeptide spacer comprises an amino acid sequence having a percentage sequence identity to the amino acid sequence of SEQ ID NO:02 of at least 90%, 91%, 92%, 93%, 94%, 95%, 96%, 97%, 98%, or 99%/o or within a range defined by any two of the aforementioned percentages. In some embodiments, the polypeptide spacer comprises or consists of the amino acid sequence of SEQ ID NO:02.

In some embodiments, a C-terminus of the ligand binding domain is joined to an N-terminus of the polypeptide spacer with no more than 20, 19, 18, 17, 16, 15, 14, 13, 12, 11, 10, 9, 8, 7, 6, 5, 4, 3, 2, 1, or 0 intervening amino acid residues. In some embodiments, the C-terminus of the ligand binding domain is joined via a peptide bond to the N-terminus of the polypeptide spacer with no intervening amino acid residues. In some embodiments, the C-terminus of the polypeptide spacer is the C-terminus of an amino acid sequence encoded by a nucleotide sequence having a percentage sequence identity to the nucleotide sequence of SEQ ID NO:01 of at least 90%, 91%, 92%, 93%, 94%, 95%, 96%, 97%, 98%, or 99% or within a range defined by any two of the aforementioned percentages. In some embodiments, a N-terminus of the polypeptide spacer is the N-terminus of an amino acid sequence having a percentage sequence identity to the amino acid sequence of SEQ ID NO:02 of at least 90%, 91%, 92%, 93%, 94%, 95%, 96%, 97%, 98%, or 99% or within a range defined by any two of the aforementioned percentages.

In some embodiments, the CAR comprises a transmembrane domain. In some embodiments, the transmembrane domain comprises a CD28 transmembrane domain. In some embodiments, the CD28 transmembrane domain comprises an amino acid sequence having a percentage sequence identity to the amino acid sequence of SEQ ID NO:04 of at least 90%, 91%, 92%, 93%, 94%, 95%, 96%, 97%, 98%, or 99% or within a range defined by any two of the aforementioned percentages. In some embodiments, the CD28 transmembrane domain comprises or consists of the amino acid sequence of SEQ ID NO:04.

In some embodiments, a C-terminus of the polypeptide spacer is joined to an N-terminus of the transmembrane domain with no more than 20, 19, 18, 17, 16, 15, 14, 13, 12, 11, 10, 9, 8, 7, 6, 5, 4, 3, 2, 1, or 0 intervening amino acid residues. In some embodiments, the C-terminus of the polypeptide spacer is joined via a peptide bond to the N-terminus of the transmembrane domain with no intervening amino acid residues. In some embodiments, the C-terminus of the polypeptide spacer is the C-terminus of an amino acid sequence having a percentage sequence identity to the amino acid sequence of SEQ ID NO:02 of at least 90%, 91%, 92%, 93%, 94%, 95%, 96%, 97%, 98%, or 99% or within a range defined by any two of the aforementioned percentages. In some embodiments, the N-terminus of the polypeptide spacer is the N-terminus of an amino acid sequence having a percentage sequence identity to the amino acid sequence of SEQ ID NO:04 of at least 90%, 91%, 92%, 93%, 94%, 95%, 96%, 97%, 98%, or 990% or within a range defined by any two of the aforementioned percentages.

In some embodiments, the CAR comprises an intracellular signalling domain. In some embodiments, the intracellular signalling domain comprises a costimulatory domain selected from the group consisting of CD27, CD28, 4-1 BB, OX-40, CD30, CD40, PD-1, ICOS, LFA-1, CD2, CD7, NKG2C, and B7-H3. In some embodiments, the intracellular signalling domain also includes a CD3-zeta domain or functional portion thereof. In some embodiments, the intracellular signalling domain the intracellular signalling domain comprises a 4-1BB costimulatory domain. In some embodiments, the 4-1BB costimulatory domain is encoded by a nucleotide sequence having a percentage sequence identity to the nucleotide sequence of SEQ ID NO:07 of at least 90%, 91%, 92%, 93%, 94%, 95%, 96%, 97%, 98%, or 99% or within a range defined by any two of the aforementioned percentages. In some embodiments, the 4-1 BB costimulatory domain is encoded by the nucleotide sequence of SEQ ID NO:07. In some embodiments, the CD3-zeta domain or functional portion thereof is encoded by a nucleotide sequence having a percentage identity to the nucleotide sequence of SEQ ID NO:08 of at least 90%, 91%, 92%, 93%, 94%, 95%, 96%, 97%, 98%, or 99% or within a range defined by any two of the aforementioned percentages. In some embodiments, the CD3-zeta domain or functional portion thereof comprises or consists of a sequence encoded by the nucleotide sequence of SEQ ID NO:08.

In some alternatives, which may include a third generation of CARs, the intracellular signalling domain also comprises a CD28 cytoplasmic domain. In some embodiments the CD28 cytoplasmic domain comprises an amino acid sequence having a sequence percentage identity to the amino acid sequence of SEQ ID NO:06 of at least 90°/%, 91%, 92%, 93%, 94%, 95%, 96%, 97%, 98%, or 99% or within a range defined by any two of the aforementioned percentages. In some embodiments, the CD28 cytoplasmic domain comprises or consists of the amino acid sequence of SEQ ID NO:06. In some embodiments the CD28 cytoplasmic domain comprises an amino acid sequence having an LL>GG substitution. In some embodiments the CD28 cytoplasmic domain comprises an amino acid sequence having a sequence percentage identity to the amino acid sequence of SEQ ID NO:18 of at least 90%, 91%, 92%, 93%, 94%, 95%, 96%, 97%, 98%, or 99% or within a range defined by any two of the aforementioned percentages. In some embodiments, the CD28 cytoplasmic domain comprises or consists of the amino acid sequence of SEQ ID NO:18. In some embodiments, which may include a second generation CAR, the intracellular signalling domain lacks a CD28 cytoplasmic domain.

Some embodiments also include a constitutive promoter operably linked to the polynucleotide encoding a chimeric antigen receptor. In some embodiments, the constitutive promoter comprises an EF1α promoter. Some embodiments also include an inducible promoter operably linked to the polynucleotide encoding a chimeric antigen receptor.

Some embodiments also include a polynucleotide encoding a cell-surface selectable marker. In some embodiments, the cell-surface selectable marker is selected from a truncated EGFR polypeptide (EGFRt) or a truncated Her2 polypeptide (Her2t).

Some embodiments also include a ribosome skip sequence located between a polynucleotide encoding the intracellular signalling domain and the polynucleotide encoding a cell-surface selectable marker. In some embodiments, the ribosome skip sequence is selected from the group consisting of a P2A sequence, a T2A sequence, an E2A sequence, and an F2A sequence.

Some embodiments also include a polynucleotide encoding a suicide gene system. In some embodiments, the suicide gene system is selected from a herpes simplex virus thymidine kinase/ganciclovir (HSVTK/GCV) suicide gene system, or an inducible caspase suicide gene system.

Some embodiments of the methods and compositions provided herein include vectors comprising any one of the nucleic acids disclosed herein encoding an anti-CD171 CAR, such as any one of the foregoing nucleic acids. In some embodiments, the vector is selected from the group consisting of a viral vector, a transposon vector, an integrase vector, and an mRNA vector. In some embodiments, the viral vector is selected from the group consisting of a lentiviral vector, a foamy viral vector, a retroviral vector, and a gamma retroviral vector. In some embodiments, the viral vector is a lentiviral vector.

Some embodiments of the methods and compositions provided herein include a polypeptide encoded by any one of the nucleic acids disclosed herein. TABLE 2 lists example sequences useful with certain embodiments provided herein.

TABLE 2

| Feature | |
|---|---|
| SEQ ID NO | Sequence |
| CE7 scFV | ATGCTGCTGCTGGTGACCAGCCTGCTGCTGTGCGAGCTGCCCC |
| SEQ ID NO: 01 | ACCCCGCCTTTCTGCTGATCCCCCAGGTGCAGCTGCAGCAGCC |
| | TGGCGCCGAGCTGGTGAAGCCAGGCGCCAGCGTGAAGCTGTC |
| | CTGCAAGGCCAGCGGCTACACCTTCACCGGCTACTGGATGCAC |
| | TGGGTGAAGCAGAGACCCGGCCACGGCCTGGAATGGATCGGC |
| | GAGATCAACCCCAGCAACGGCCGGACCAACTACAACGAGCGG |
| | TTCAAGAGCAAGGCCACCCTGACCGTGGACAAGAGCAGCACC |
| | ACCGCCTTCATGCAGCTGTCCGGCCTGACCAGCGAGGACAGC |
| | GCCGTGTACTTCTGCGCCAGGGACTACTACGGCACCAGCTACA |
| | ACTTCGACTACTGGGGCCAGGGCACCACACTGACCGTGAGCA |
| | GCGGCGGAGGGGGCTCTGGCGGCGGAGGATCTGGGGGAGGG |
| | GGCAGCGACATCCAGATGACCCAGAGCAGCAGCAGCTTCAGC |
| | GTGAGCCTGGGCGACCGGGTGACCATCACCTGTAAGGCCAAC |
| | GAGGACATCAACAACCGGCTGGCCTGGTATCAGCAGACCCCC |
| | GGCAACAGCCCCAGGCTGCTGATCAGCGGCGCCACCAACCTG |
| | GTGACCGGCGTGCCCAGCCGGTTTAGCGGCAGCGGCTCCGGC |
| | AAGGACTACACCCTGACCATCACAAGCCTGCAGGCCGAGGAC |
| | TTCGCCACCTACTACTGCCAGCAGTACTGGTCCACCCCCTTCA |
| | CCTTCGGCAGCGGCACCGAGCTGGAAA |
| L (long) spacer | ESKYGPPCPPCPAPEFDGGPSVFLFPPKPKDTLMISRTPEVTCVVV |
| L235D | DVSQEDPEVQFNWYVDGVEVHNAKTKPREEQFNSTYRVVSVLT |
| substitution | VLHQDWLNGKEYKCKVSNKGLPSSIEKTISKAKGQPREPQVYTL |
| (underlined) | PPSQEEMTKNQVSLTCLVKGFYPSDIAVEWESNGQPENNYKTTPP |
| SEQ ID NO: 02 | VLDSDGSFFLYSRLTVDKSRWQEGNVFSCSVMHEALHNHYTQK |
| | SLSLSLGK |
| L (long) spacer | GAATCTAAGTACGGACCGCCCTGCCCCCGAGTTCGACGGCGG |
| L235D codon | ACCCAGCGTGTTCCTGTTCCCCCCCAAGCCCAAGGACACCCTG |
| substitution | ATGATCAGCCGGACCCCCGAGGTGACCTGCGTGGTGGTGGAC |
| (underlined) | GTGAGCCAGGAAGATCCCGAGGTCCAGTTCAATTGGTACGTG |
| SEQ ID NO: 03 | GACGGCGTGGAAGTGCACAACGCCAAGACCAAGCCCAGAGA |
| | GGAACAGTTCAACAGCACCTACCGGGTGGTGTCTGTGCTGACC |
| | GTGCTGCACCAGGACTGGCTGAACGGCAAAGAATACAAGTGC |
| | AAGGTGTCCAACAAGGGCCTGCCCAGCAGCATCGAAAAGACC |
| | ATCAGCAAGGCCAAGGGCCAGCCTCGCGAGCCCCAGGTGTAC |
| | ACCCTGCCTCCCTCCCAGGAAGAGATGACCAAGAACCAGGTG |
| | TCCCTGACCTGCCTGGTGAAGGGCTTCTACCCCAGCGACATCG |
| | CCGTGGAGTGGGAGAGCAACGGCCAGCCTGAGAACAACTACA |
| | AGACCACCCCTCCCGTGCTGGACAGCGACGGCAGCTTCTTCCT |
| | GTACAGCCGGCTGACCGTGGACAAGAGCCGGTGGCAGGAAGG |
| | CAACGTCTTTAGCTGCAGCGTGATGCACGAGGCCCTGCACAAC |
| | CACTACACCCAGAAGAGCCTGAGCCTGTCCCTGGGCAAG |
| CD28tm | MFWVLVVVGGVLACYSLLVTVAFIIFWV |
| SEQ ID NO: 04 | |
| CD28tm | ATGTTCTGGGTGCTGGTGGTGGTCGGAGGCGTGCTGGCCTGCT |
| SEQ ID NO: 05 | ACAGCCTGCTGGTCACCGTGGCCTTCATCATCTTTTGGGTG |
| CD28 | RSKRSRLLHSDYMNMTPRRPGPTRKHYQPYAPPRDFAAYRS |
| cytoplasmic | |
| SEQ ID NO: 06 | |
| 41-BB | AAACGGGGCAGAAAGAAACTCCTGTATATATTCAAACAACCA |
| SEQ ID NO: 07 | TTTATGAGACCAGTACAAACTACTCAAGAGGAAGATGGCTGT |
| | AGCTGCCGATTTCCAGAAGAAGAAGAAGGAGGATGTGAACTG |
| CD3ζ | CGGGTGAAGTTCAGCAGAAGCGCCGACGCCCCTGCCTACCAG |
| SEQ ID NO: 08 | CAGGGCCAGAATCAGCTGTACAACGAGCTGAACCTGGGCAGA |
| | AGGGAAGAGTACGACGTCCTGGATAAGCGGAGAGGCCGGGA |
| | CCCTGAGATGGGCGGCAAGCCTCGGCGGAAGAACCCCCAGGA |
| | AGGCCTGTATAACGAACTGCAGAAAGACAAGATGGCCGAGGC |
| | CTACAGCGAGATCGGCATGAAGGGCGAGCGGAGGCGGGGCA |
| | AGGGCCACGACGGCCTGTATCAGGGCCTGTCCACCGCCACCA |
| | AGGATACCTACGACGCCCTGCACATGCAGGCCCTGCCCCCAA |
| | GG |
| T2A | CTCGAGGGCGGCGGAGAGGGCAGAGGAAGTCTTCTAACATGC |
| SEQ ID NO: 09 | GGTGACGTGGAGGAGAATCCCGGCCCTAGG |
| EGFRt | CGCAAAGTGTGTAACGGAATAGGTATTGGTGAATTTAAAGAC |
| SEQ ID NO: 10 | TCACTCTCCATAAATGCTACGAATATTAAACACTTCAAAAACT |
| | GCACCTCCATCAGTGGCGATCTCCACATCCTGCCGGTGGCATT |
| | TAGGGGTGACTCCTTCACACATACTCCTCCTCTGGATCCACAG |
| | GAACTGGATATTCTGAAAACCGTAAAGGAAATCACAGGGTTT |
| | TTGCTGATTCAGGCTTGGCCTGAAAACAGGACGGACCTCCATG |
| | CCTTTGAGAACCTAGAAATCATACGCGGCAGGACCAAGCAAC |
| | ATGGTCAGTTTTCTCTTGCAGTCGTCAGCCTGAACATAACATC |
| | CTTGGGATTACGCTCCCTCAAGGAGATAAGTGATGGAGATGT |
| | GATAATTTCAGGAAACAAAAATTTGTGCTATGCAAATACAAT |
| | AAACTGGAAAAAACTGTTTGGGACCTCCGGTCAGAAAACCAA |
| | AATTATAAGCAACAGAGGTGAAAACAGCTGCAAGGCCACAGG |
| | CCAGGTCTGCCATGCCTTGTGCTCCCCCGAGGGCTGCTGGGGC |
| | CCGGAGCCCAGGGACTGCGTCTCTTGCCGGAATGTCAGCCGA |
| | GGCAGGGAATGCGTGGACAAGTGCAACCTTCTGGAGGGTGAG |
| | CCAAGGGAGTTTGTGGAGAACTCTGAGTGCATACAGTGCCAC |
| | CCAGAGTGCCTGCCTCAGGCCATGAACATCACCTGCACAGGA |
| | CGGGGACCAGACAACTGTATCCAGTGTGCCCACTACATTGAC |
| | GGCCCCCACTGCGTCAAGACCTGCCCGGCAGGAGTCATGGGA |
| | GAAAACAACACCCTGGTCTGGAAGTACGCAGACGCCGGCCAT |
| | GTGTGCCACCTGTGCCATCCAAACTGCACCTACGGATGCACTG |
| | GGCCAGGTCTTGAAGGCTGTCCAACGAATGGGCCTAAGATCC |
| | CGTCCATCGCCACTGGGATGGTGGGGGCCCTCCTCTTGCTGCT |
| | GGTGGTGGCCCTGGGGATCGGCCTCTTCATG |
| S (small) spacer | ESKYGPPCPPCP |
| SEQ ID NO: 11 | |
| S (small) spacer | GAATCTAAGTACGGACCGCCCTGCCCCCCTTGCCCT |
| SEQ ID NO: 12 | |
| M (medium) | ESKYGPPCPPCPGQPREPQVYTLPPSQEEMTKNQVSLTCLVKGFY |
| spacer | PSDIAVEWESNGQPENNYKTTPPVLDSDGSFFLYSRLTVDKSRW |
| SEQ ID NO: 13 | QEGNVFSCSVMHEALHNHYTQKSLSLSLGK |
| M (medium) | GAATCTAAGTACGGACCGCCCTGCCCCCCTTGCCCTGGCCAGC |
| spacer | CTAGAGAACCCCAGGTGTACACCCTGCCTCCCAGCCAGGAAG |
| SEQ ID NO: 14 | AGATGACCAAGAACCAGGTGTCCCTGACCTGCCTGGTCAAAG |
| | GCTTCTACCCCAGCGATATCGCCGTGGAATGGGAGAGCAACG |
| | GCCAGCCCGAGAACAACTACAAGACCACCCCCCCTGTGCTGG |
| | ACAGCGACGGCAGCTTCTTCCTGTACTCCCGGCTGACCGTGGA |
| | CAAGAGCCGGTGGCAGGAAGGCAACGTCTTCAGCTGCAGCGT |
| | GATGCACGAGGCCCTGCACAACCACTACACCCAGAAGTCCCT |
| | GAGCCTGAGCCTGGGCAAG |
| L (long) spacer | ESKYGPPCPPCPAPEFLGGPSVFLFPPKPKDTLMISRTPEVTCVVV |
| SEQ ID NO: 15 | DVSQEDPEVQFNWYVDGVEVHNAKTKPREEQFNSTYRVVSVLT |
| | VLHQDWLNGKEYKCKVSNKGLPSSIEKTISKAKGQPREPQVYTL |
| | PPSQEEMTKNQVSLTCLVKGFYPSDIAVEWESNGQPENNYKTTPP |
| | VLDSDGSFFLYSRLTVDKSRWQEGNVFSCSVMHEALHNHYTQK |
| | SLSLSLGK |
| L (long) spacer | GAATCTAAGTACGGACCGCCCTGCCCCCCTTGCCCTGCCCCCG |
| SEQ ID NO: 16 | AGTTCGACGGCGGACCCAGCGTGTTCCTGTTCCCCCCCAAGCC |
| | CAAGGACACCCTGATGATCAGCCGGACCCCCGAGGTGACCTG |
| | CGTGGTGGTGGACGTGAGCCAGGAAGATCCCGAGGTCCAGTT |
| | CAATTGGTACGTGGACGGCGTGGAAGTGCACAACGCCAAGAC |
| | CAAGCCCAGAGAGGAACAGTTCCAGAGCACCTACCGGGTGGT |
| | GTCTGTGCTGACCGTGCTGCACCAGGACTGGCTGAACGGCAA |
| | AGAATACAAGTGCAAGGTGTCCAACAAGGGCCTGCCCAGCAG |
| | CATCGAAAAGACCATCAGCAAGGCCAAGGGCCAGCCTCGCGA |
| | GCCCCAGGTGTACACCCTGCCTCCCTCCCAGGAAGAGATGACC |
| | AAGAACCAGGTGTCCCTGACCTGCCTGGTGAAGGGCTTCTACC |
| | CCAGCGACATCGCCGTGGAGTGGGAGAGCAACGGCCAGCCTG |
| | AGAACAACTACAAGACCACCCCTCCCGTGCTGGACAGCGACG |
| | GCAGCTTCTTCCTGTACAGCCGGCTGACCGTGGACAAGAGCCG |
| | GTGGCAGGAAGGCAACGTCTTTAGCTGCAGCGTGATGCACGA |
| | GGCCCTGCACAACCACTACACCCAGAAGAGCCTGAGCCTGTC |
| | CCTGGGCAAG |
| CD28 | CGGAGCAAGCGGAGCAGAGGCGGCCACAGCGACTACATGAA |
| cytoplasmic | CATGACCCCCAGACGGCCTGGCCCCACCCGGAAGCACTACCA |
| (LL->GG, at 7^{th} | GCCCTACGCCCCACCCAGGGACTTTGCCGCCTACAGAAGC |
| and 8^{th} amino | |
| acid residues) | |
| SEQ ID NO: 17 | |
| CD28 | RSKRSRGGHSDYMNMTPRRPGPTRKHYQPYAPPRDFAAYRS |
| cytoplasmic | |
| (LL->GG, at 7^{th} | |
| and 8^{th} residues) | |
| SEQ ID NO: 18 | |
| L (long) spacer | ESKYGPPCPPCPAPEFDGGPSVFLFPPKPKDTLMISRTPEVTCVVV |
| L235D and | DVSQEDPEVQFNWYVDGVEVHNAKTKPREEQFQSTYRVVSVLT |
| N257Q | VLHQDWLNGKEYKCKVSNKGLPSSIEKTISKAKGQPREPQVYTL |
| substitutions | PPSQEEMTKNQVSLTCLVKGFYPSDIAVEWESNGQPENNYKTTPP |
| (underlined) | VLDSDGSFFLYSRLTVDKSRWQEGNVFSCSVMHEALHNHYTQK |
| SEQ ID NO: 19 | SLSLSLGK |
| L (long) spacer | GAATCTAAGTACGGACCGCCCTGCCCCCGAGTTCGACGGCGG |
| L235D and | ACCCAGCGTGTTCCTGTTCCCCCCCAAGCCCAAGGACACCCTG |
| N257Q codon | ATGATCAGCCGGACCCCCGAGGTGACCTGCGTGGTGGTGGAC |
| substitutions | GTGAGCCAGGAAGATCCCGAGGTCCAGTTCAATTGGTACGTG |
| (underlined) | GACGGCGTGGAAGTGCACAACGCCAAGACCAAGCCCAGAGA |
| SEQ ID NO: 20 | GGAACAGTTCCAGAGCACCTACCGGGTGGTGTCTGTGCTGAC |
| | CGTGCTGCACCAGGACTGGCTGAACGGCAAAGAATACAAGTG |
| | CAAGGTGTCCAACAAGGGCCTGCCCAGCAGCATCGAAAAGAC |
| | CATCAGCAAGGCCAAGGGCCAGCCTCGCGAGCCCCAGGTGTA |
| | CACCCTGCCTCCCTCCCAGGAAGAGATGACCAAGAACCAGGT |
| | GTCCCTGACCTGCCTGGTGAAGGGCTTCTACCCCAGCGACATC |
| | GCCGTGGAGTGGGAGAGCAACGGCCAGCCTGAGAACAACTAC |
| | AAGACCACCCCTCCCGTGCTGGACAGCGACGGCAGCTTCTTCC |
| | TGTACAGCCGGCTGACCGTGGACAAGAGCCGGTGGCAGGAAG |
| | GCAACGTCTTTAGCTGCAGCGTGATGCACGAGGCCCTGCACA |
| | ACCACTACACCCAGAAGAGCCTGAGCCTGTCCCTGGGCAAG |

### Certain Cells

Some embodiments of the methods and compositions provided herein include host cells comprising any one of the nucleic acids provided herein. In some embodiments, the host cell is a CD4+ T-cell or a CD8+ T-cell. In some embodiments, the host cell is a precursor T-cell, or a hematopoietic stem cell. In some embodiments, the host cell is a CD8+ cytotoxic T-cell selected from the group consisting of a naïve CD8+ T-cell, a CD8+ memory T-cell, a central memory CD8+ T-cell, a regulatory CD8+ T-cell, an IPS derived CD8+ T-cell, an effector memory CD8+ T-cell, and a bulk CD8+ T-cell. In some embodiments, the host cell is a CD4+ T helper cell selected from the group consisting of a naïve CD4+ T-cell, a CD4+ memory T-cell, a central memory CD4+ T-cell, a regulatory CD4+ T-cell, an IPS derived CD4+ T-cell, an effector memory CD4+ T-cell, and a bulk CD4+ T-cell. In some embodiments, the cell is allogenic to a subject, preferably a human, and in other embodiments, the cell is autologous to a subject, preferably a human.

Some embodiments of the methods and compositions provided herein include pharmaceutical compositions comprising any one of the host cells provide herein and a pharmaceutically acceptable excipient.

### Certain Therapeutic Methods

Some of the methods and compositions provided herein relate to therapeutic alternatives. Some such methods include treating, inhibiting or ameliorating a cancer in a subject, preferably a human, comprising administering any one of the host cells provided herein to the subject. In some embodiments, the subject is mammalian. In some embodiments, the subject, preferably a human, is selected or identified to receive a CAR specific for the cancer said subject is experiencing. Such selection or identification of a subject or population of subjects responsive to such a therapy can be made by a clinician or physician using clinical and/or diagnostic evaluation e.g., diagnostic evaluation of the presence or amount of CD171 on cancer cells residing in said subject.

In some embodiments, the subject is administered a dose comprising a plurality of the host cell sufficient to treat, inhibit or ameliorate the cancer that is less than a dose sufficient to treat, inhibit or ameliorate the cancer of a plurality of cells comprising a CAR comprising a polypeptide spacer having a length less than 120 consecutive amino acid residues.

In some embodiments, the subject is administered a dose comprising a plurality of the host cell of less than 1×10⁶ cell/kg, 2×10⁶ cell/kg, 3×10⁶ cell/kg, 4×10⁶ cell/kg, 5×10⁶ cell/kg, 6×10⁶ cell/kg, 7×10⁶ cell/kg, 8×10⁶ cell/kg, 9×10⁶ cell/kg, but not less than zero.

Some embodiments also include administering cetuximab to the subject in addition to one or more of the therapies disclosed herein.

In some embodiments, the cancer comprises a CD171 expressing cell. In some embodiments, the cancer comprises a brain cancer. In some embodiments, the cancer is selected from a neuroblastoma or a ganglioneuroblastoma. Additional embodiments include the use of any one or more of the host cells described herein as a medicament, preferably a medicament for the treatment, inhibition, or amelioration of a cancer, such as a neuroblastoma or a ganglioneuroblastoma.

### EXAMPLES

### Example 1 (Comparative Example)—In Vitro and Xenograft Activities of Anti-CD171 CARs

Anti-CD171 CARs were prepared by methods substantially similar to those described in U.S. 2018/0009891 and in Kunkele A. et al., (2015) Cancer Immunol Res 3:368-379 which are each expressly incorporated by reference in its entirety. The anti-CD171 CARs included a second generation of CARs having a short spacer (IgG4 hinge domain), a medium spacer (IgG4 hinge-CH2 domain), or a long spacer (IgG4-CH2-CH3 domain). FIG. 1 depicts a schematic of an exemplary structure of a nucleic acid encoding a second generation of CARs and a third generation anti-CD171 CAR with a short spacer. Second generation CARs lacked an intracellular signalling domain comprising a CD28 cytoplasmic domain. Third generation CARs included an intracellular signalling domain comprising a CD28 cytoplasmic domain located between a CD28 transmembrane domain and a 4-1BB domain.

In vitro studies and in vivo xenograft studies were performed to determine the activity of cells containing each generation of CAR. See Kunkele A. et al., (2015) Cancer Immunol Res 3:368-379. Briefly, CARs with a longer spacer induced higher levels of phospho-ERK upon co-culture with CD171+Be2 neuroblastoma cells at a 1:1 E:T ratio than CARs with a medium or a short spacer. CARs with a longer spacer induced higher levels of CD137 surface expression upon co-culture with CD171+Be2 cells at a 1:1 E:T ratio than CARs with a medium or a short spacer. CARs with a longer spacer induced higher levels of specific lysis upon co-culture with CD171+Be2 cells than CARs with a medium or a short spacer. CARs with a longer spacer stimulated high levels of cytokine secretion, IL-2, TNFalpha, and IFN gamma in mixed tumor cultures than CARs with a medium or a short spacer.

However, anti-CD171 CARs with a longer spacer had substantially less activity in vitro than CARs with medium or shorter spacers. Briefly, an intracranial mouse neuroblastoma xenograft therapy model was used in which ffLuc+Be2 tumors were stereotacticly implanted at day 0, and CARs were administered at day 7. In tumors contacted with CARs having a long spacer, increasing signal from tumors and mouse survival was substantially similar to tumors contacted with control cells. In contrast, when tumors were contacted with CARs having a short or medium spacer, mouse survival was substantially increased compared to tumors contacted with control cells. Thus, unexpectedly, CAR constructs having a long spacer generated the highest in vitro activity but exhibited attenuated antitumor potency in vivo.

### Example 2—In Vitro and Xenograft Activities of Anti-CD171 CARs

Second and third generation anti-CD171 CARs containing a double mutant long spacer were generated in which the double mutant long spacer included an IgG4-CH2-CH3 domain in which the CH2 domain included an L235D substitution and an N257Q substitution (SEQ ID NO:18). The L235D substitution reduced or removed binding to human FcγR1, and the N257Q substitution reduced or removed binding to murine FcγR1.

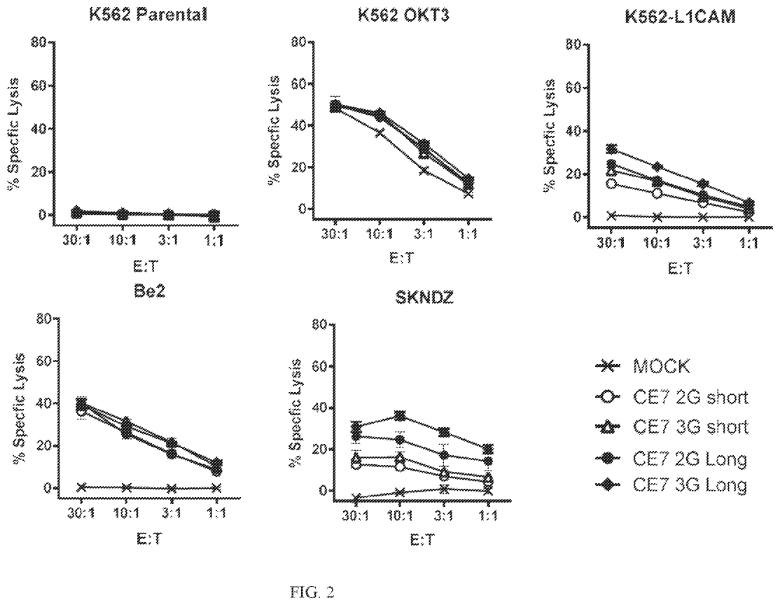

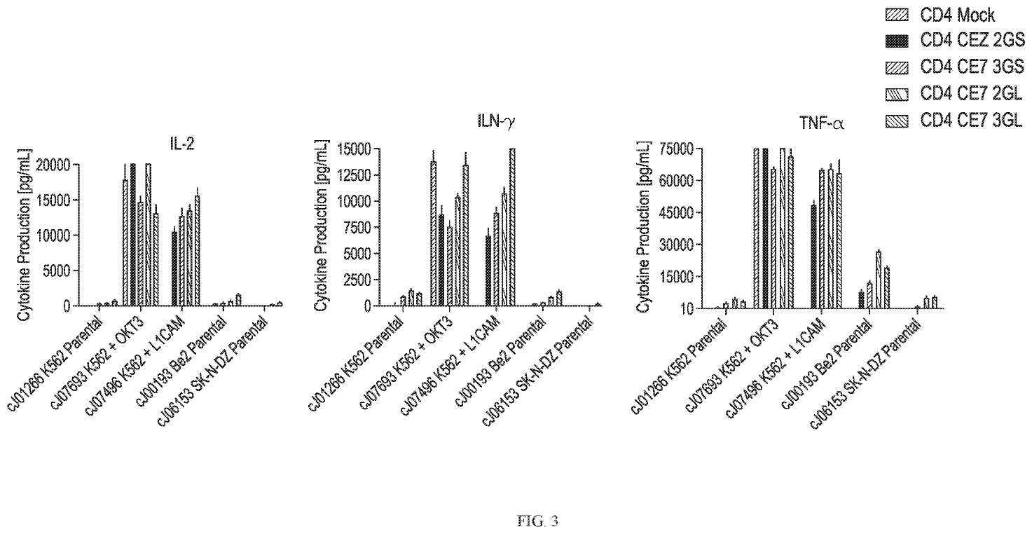

In vitro studies and in vivo xenograft studies were performed to compare the activities of the following CARs: a second generation CAR with a short spacer; a third generation CAR with a short space; a second generation CAR with a double mutant long spacer; and a third generation CAR with a double mutant long spacer. Briefly, cells containing CARs were co-cultured in vitro at various ratios with control cells or with CD171+ target cells, and specific lysis of the target cells was measured (FIG. 2). Specific lysis was observed against CD171+ target Be2 cells with each CAR, with CARs having long spacers having some greater activity than CARs having short spacers. Cells containing CARs were co-cultured in vitro with control cells or with CD171+ target cells, and stimulated cytokines were measured (FIG. 3). CARs induced cytokine stimulation with CARs having long spacers having some greater activity than CARs having short spacers.

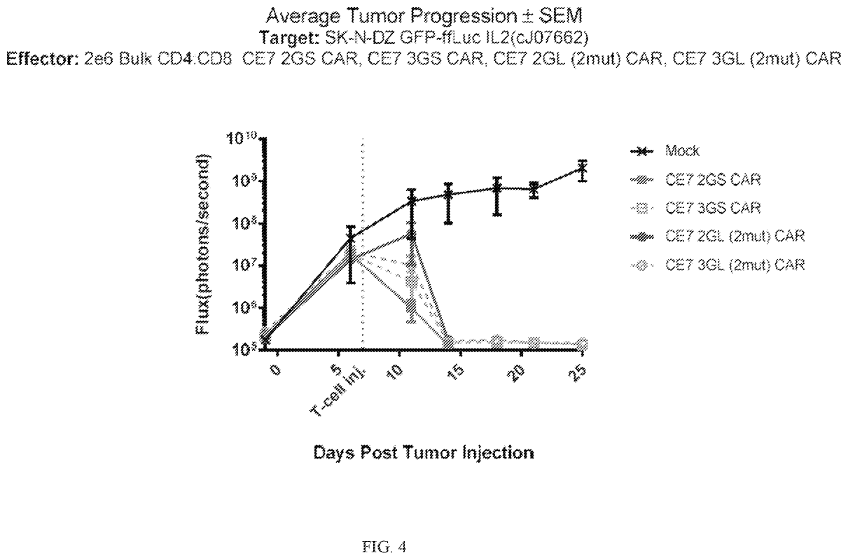

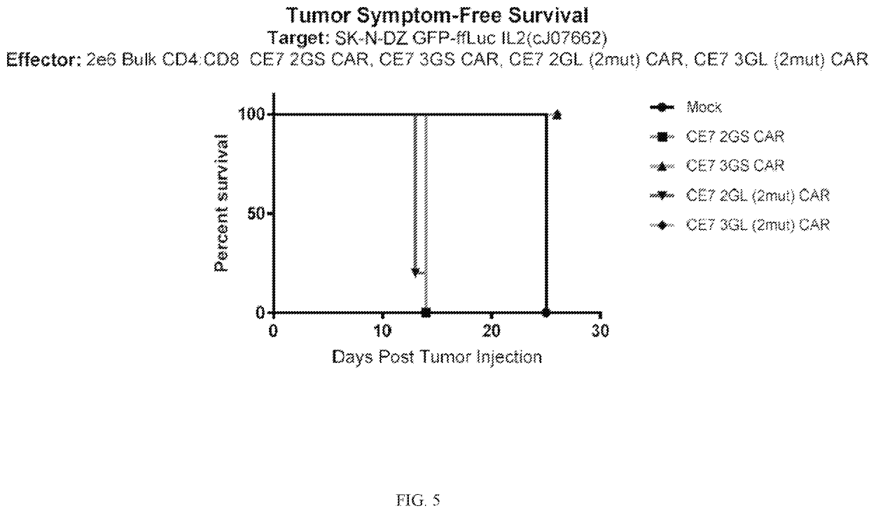

An in vivo intracranial mouse neuroblastoma xenograft therapy model was used in which ffLuc+Be2 tumors were stereotacticly implanted at day 0, and CARs were administered at day 7. Third generation CARs with long or short spacers eradicated tumors (FIG. 4), and prolonged survival (FIG. 5). Second generation CARs with short or long spacers also eradicated tumors but resulted in earlier death (FIG. 4 and FIG. 5).

### Example 3—Preparation of CARs for Infusion

Anti-CD171 CARs were prepared by methods substantially similar to those described in U.S. 2018/0009891 which is expressly incorporated by reference in its entirety. The CARs included a second generation anti-CD171 CAR comprising a short spacer; a second generation anti-CD171 CAR comprising a long spacer; and a third generation anti-CD171 CAR comprising a short spacer. The short spacer included an IgG4 hinge domain. The long spacer included an IgG4 hinge-CH2-CH3 domain in which the CH2 domain included an L235D substitution (SEQ ID NO:02). The L235D substitution reduced or removed binding to FcγR1.

### Example 4—Preparation of CAR T Cells for Infusion

CAR T cells from subjects were prepared by methods substantially similar to those described in Kunkele A., et al., (2017) Clin Cancer Res 23:466-477, which is expressly incorporated by reference in its entirety. Briefly, subjects underwent standard apheresis for collection of approximately 5×10⁹ PBMCs. After isolation of a CD4+ and a CD8+ T-cell population using CD4 and CD8 magnetic beads, cells were stimulated with anti-CD3/CD28 beads (Life Technologies) and cultured in X-Vivo media (Lonza) supplemented with 10% defined, irradiated, heat-inactivated FBS (GE Hyclone), IL15 (0.5 ng/mL), and for CD8+ cells IL2 (50 U/mL), for CD4+ cells IL7 (5 ng/mL). Transduction with the CE7 CAR lentiviral vector was performed on day 1 by centrifugation at 800×g for 30 minutes at 32° C. with lentiviral supernatant [approximate multiplicity of infection (MOI)=0.1] supplemented with 1 mg/mL protamine sulfate (APP Pharmaceuticals). Residual stimulation beads were removed from culture prior to cryopreservation using the CTS magnet system (Life Technologies). Cells were expanded and cryopreserved in CryoStor CS-5 (Sigma) until the day of planned infusion.

### Example 5—Phase I Clinical Trial

### Trial Protocol

A Phase 1 clinical trial was initiated to determine the feasibility and safety of cellular immunotherapy for recurrent/refractory neuroblastoma using autologous T-cells lentivirally transduced to express CD171-specific chimeric antigen receptors.

Subject inclusion criteria included: prior diagnosis of neuroblastoma or ganglioneuroblastoma either by histologic verification and/or demonstration of tumor cells in the bone marrow with increased catecholamine levels; male or female subjects ≤26 years of age; diagnosis of high risk neuroblastoma at initial diagnosis or if non-high risk at time of initial diagnosis must have had evidence of metastatic progression when >18 months of age; measurable or evaluable disease; Lansky or Karnofsky performance status score of ≥50; life expectancy of ≥8 weeks; recovered from significant acute toxic effects of all prior chemotherapy, immunotherapy, or radiotherapy prior to enrollment onto this study; ≥7 days since last chemotherapy or biologic therapy administration; no systemic corticosteroids (unless physiologic replacement dosing) within 7 days of enrollment; ≥3 half-lives or 30 days from time of last dose of anti-tumor directed antibody therapy, whichever is shorter from time of enrollment; ≥6 weeks from myeloablative therapy and autologous stem cell transplant (timed from stem cell infusion), patients who received stem cell infusion following non-myeloablative therapy are eligible once they meet all other eligibility requirements, and patient must not have received a prior allogeneic hematopoietic stem cell transplant; no prior genetically modified cell therapy that is still detectable or prior virotherapy; must not be receiving external beam radiation therapy at the time of study enrollment. ≥12 weeks from prior I131 MIBG therapy; adequate organ function; adequate laboratory values; and negative HIV antigen and antibody, Hepatitis B surface antigen and Hepatitis C antibody within 3 months prior to enrollment, for patients with positive Hepatitis C antibody, negative PCR testing must be documented in order to be eligible.

Subject exclusion criteria included: history of relevant CNS pathology or current relevant CNS pathology (non-febrile seizure disorder requiring ongoing anti-epileptic medications, paresis, aphasia, cerebrovascular ischemia/hemorrhage, severe brain injuries, dementia, cerebellar disease, organic brain syndrome, psychosis, coordination or movement disorder), and patients may have CNS intracranial tumor; pregnant or breast-feeding; unable to tolerate apheresis procedure including placement of temporary apheresis catheter if necessary; presence of active malignancy other than neuroblastoma; presence of known intracranial metastatic neuroblastoma, and skull based disease with soft tissue extension is allowed; presence of active severe infection; presence of any concurrent medical condition that, in the opinion of the protocol principal investigator, would prevent the patient from undergoing protocol-based therapy; presence of a primary immunodeficiency/bone marrow failure syndrome; receiving any other anti-cancer agents or radiotherapy at the time of study entry; and unwilling or unable to provide consent/assent for participation in the study and 15-year follow-up.

The protocol used was substantially the same as the following. Upon meeting the eligibility requirements and enrolling on study, subjects underwent apheresis to obtain the T cells for the generation of the CD171 CAR+ T cells. The T cells were isolated from the apheresis product, the CD4 and CD8 T cells were then selected and grown separately, transduced with a lentivirus to express the CD171 CAR, as well as, a truncated EGFR (EGFRt) that had no signaling capacity and expanded in culture over a 4-6 week period. After the CAR+ T cells had been generated, the subject underwent a disease assessment and determination of necessary lymphodepletion therapy. At least 48 hours after the completion of lymphodepletion, the subject received an infusion of CAR+ T cells at an approximate 1:1 ratio of CD4 to CD8 CAR+ T cells. Some subjects further received cetuximab for ablation of the genetically modified T cells. Criteria to receive cetuximab included acute toxicities that were life threatening, as well as, studies indicating lymphoproliferative disorder arising from an infused genetically modified T cell. Primary outcome measure included dose limiting toxicity (DLT) in which patients were evaluated through day 28 for occurrence of DLT. Secondary outcome measures included tumor response in which patients were evaluated by a revised international neuroblastoma response criteria with a 42 day timeframe.

The clinical trial included three experimental arms: A, B, and C. Experimental arm A included the use of short spacer second generation CE7R CAR T cells. The protocol used was substantially the same as the following. Autologous CD4 and CD8 cells were lentivirally transduced to generate patient derived CD171 specific CAR T cells, which also expressed an EGFRt. Patients received lymphodepletion chemotherapy prior to intravenous infusion with the autologous T cells transduced to express 4-1BB:zeta CD171CAR and EGFRt. The CD171 specific CAR T cells were administered approximately 2-3 days after lymphodepletion chemotherapy. Cells were administered approximately 1:1 CD4 and CD8 cells and dose levels were evaluated for a total T cell dose of: 1×10⁶ cells/kg (dose 1), 5×10⁶ cells/kg (dose 2), 1×10⁷ cells/kg (dose 3), 5×10⁶ cells/kg (dose 4), or 1×10⁸ cells/kg (dose 5).

Experimental arm B included the use of short spacer third generation CE7R CAR T cells. The protocol used was substantially the same as the following. Autologous CD4 and CD8 cells were lentivirally transduced to generate patient derived CD171 specific CAR T cells which also expressed an EGFRt. Patients received lymphodepletion chemotherapy prior to intravenous infusion of the autologous T cells transduced to express CD28:4-1BB:zeta CD171CAR and EGFRt. The CD171 specific CAR T cells were administered approximately 2-3 days after lymphodepletion chemotherapy. Cells were administered approximately 1:1 CD4 and CD8 cells and dose levels were evaluated for a total T cell dose of; 1×10⁶ cells/kg (dose 1), 5×10⁶ cells/kg (dose 2), 1×10⁷ cells/kg (dose 3), 5×10⁷ cells/kg (dose 4), or 1×10⁸ cells/kg (dose 5).

Experimental arm C included the use of long spacer second generation CE7R CAR T cells. The protocol used was substantially the same as the following. Autologous CD4 and CD8 cells were lentivirally transduced to generate patient derived CD171 specific CAR T cells which also expressed an EGFRt. Patients received lymphodepletion chemotherapy prior to intravenous infusion of autologous T cells transduced to express 4-1BB:zeta CD171CAR and EGFRt. CD171 specific CAR T cells were administered approximately 2-3 days after lymphodepletion chemotherapy. Cells were administered approximately 1:1 CD4 and CD8 cells and dose levels were evaluated for a total T cell dose of; 1×10⁶ cells/kg (dose 1), 5×10⁶ cells/kg (dose 2), 1×10⁷ cells/kg (dose 3), 5×10⁷ cells/kg (dose 4), or 1×10⁸ cells/kg (dose 5).

### Results

Twenty-eight (28) subjects were enrolled in the study. Nineteen (19) subjects received CAR T-cell infusion, two (2) of which have received a second infusion. Six (6) subjects went off protocol therapy prior to receiving CAR T cell infusion for reasons including family preference, disease progression and ineligibility for T cell infusion, or inability to manufacture CAR T cells. Three (3) subjects are clinically stable and subjects are waiting for signs/symptoms of disease progression prior to proceeding with T cell infusions.

Grade 2 maculopapular skin rash and Grade 3-4 hyponatremia were observed in subjects treated at dose level 3 arm B, and dose level 5 arm A. The subjects of dose level 5 arm A meeting criteria for dose limiting toxicity and criteria to resume the prior “k-in-row” up-and-down dose escalation statistical design. The protocol was amended to include additional evaluation of serum and urine electrolytes following CAR T cell infusion. Subsequent dose assignments were restarted at dose level 4 arm A, and dose level 2 arm B per the protocol statistical design. Since resuming “k-in-row” statistical design for arms A and B, eleven (11) patients have been enrolled and six (6) received T cell infusion in arms A and B: arm A dose level 4 (2 subjects), arm B dose level 2 (3 subjects), arm B dose level 3 (1 subject). Arm C has opened accrual in the single patient escalation design phase and one (1) subject has been treated at dose level 1 without experiencing a DLT. Of note, three (3) patients were treated on arm B dose level 2, meeting criteria to escalate back to arm B dose level 3 on which one (1) additional patient has been treated without DLT.

Toxicities related to T cells include cytokine release syndrome, skin rash and hyponatremia. Ten (10) of nineteen (19) subjects developed Grade 1-2 skin rashes after T cell infusion: 4/4 subjects on arm B dose level 2 (subjects S14, S18, S20 and S21); 2/2 subjects on arm B dose level 3 (subjects S15, S23); 3/4 subjects on arm A dose level 4 (subjects S11, S22, S25); and 1/1 subject on arm A dose level 5 (subject S16). Eight (8) of ten (10) subjects had evidence of T cell persistence in blood concurrent with skin rash. Skin biopsies were performed on subjects, S15 and S22, both of which demonstrated co-localization of an EGFRt+ T cell infiltrate and CD171 positive cells at the dermal-epidermal junction, consistent with on-target, off-tumor effects of the CAR T cells. Hyponatremia developed in two (2) subjects on arm B dose level 3 (subject S15), and arm A dose level 5 (subject S16), both with concurrent T cell persistence.

T cells were not detected in the blood of subjects receiving arm A or B at dose level 1, except for subject S06 who had detectable CAR T cells in bone marrow at Day 84. Notably, subject S27 on arm C dose level 1 had detectable CAR T cells through day 28. In contrast, T cell persistence at Day 7 has been observed in the 11/14 subjects receiving dose levels 2 or above, and in 7/8 subjects treated at dose levels 3 and above. Best overall response for the eighteen (18) subjects evaluable for response was a partial response in one (1) subject (S27) on arm C dose level 1. One patient (S06—arm B dose level 1) who developed progressive disease, has had prolonged stable disease for 3+ years since CAR T cell infusion without any subsequent therapy. Pseudo-progression, defined as increase in size of a known metastatic lesion followed by regression of that lesion, was seen in 7/19 subjects, all of whom had T cell persistence documented at a minimum of Day 7. Biopsy of skull metastatic lesion performed in subject S15 on day 13 revealed CD3+ infiltration with approximately 30%+ for EGFRt consistent with CAR T cell infiltration.

Toxicity and pseudo-progression appeared to be associated with T cell persistence, with some suggestion of dose and arm association. Two subjects have been reinfused with CAR T cells, one on arm B dose level 2 (S20) and arm C dose level 1 (S27). S20 had stable disease for ˜210 days after a first infusion, and due to asymptomatic progressive disease, a second infusion of stored CAR T cells was performed. Infusion #2 was well-tolerated, without any adverse events attributable to CAR T cells. The patient experienced asymptomatic progressive disease on Day +42 and returned to his referring institution. Subject S27 had a partial response to a first CAR T cell infusion at Day 42, as evidenced by regression of one of the subject's known metastatic lesions on MIBG. Based on these findings, the subject received a second CAR T cell infusion, which was well-tolerated and developed progressive disease 128 days after this second infusion.

Surprisingly, subject S27 treated with just a low dose (dose level 1) of CAR T cells comprising long spacer second generation CARs (arm C) demonstrated CAR T cell persistence through at least day 28. In contrast, subjects treated with higher doses (dose level 2 and greater) of CAR T cells comprising short spacer second or third generation CARs (arms A and B) generally demonstrated CAR T cell persistence just at day 7. Best overall response for the eighteen (18) subjects evaluable for response was a partial response in one (1) subject (S27) on arm C dose level 1.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A nucleic acid comprising a polynucleotide encoding a chimeric antigen receptor (CAR), wherein the CAR comprises:
a ligand binding domain capable of or configured to specifically bind to CD171;
a polypeptide spacer having a length greater than or equal to 120 and less than or equal to 230 consecutive amino acid residues;
a transmembrane domain; and
an intracellular signalling domain.

2.-45. (canceled)


SEQUENCE LISTING (20 sequences)

SEQ ID NO: 1 - DNA, 787 bp, Artificial Sequence
  CE7 scFV
atgctgctgctggtgaccagcctgctgctgtgcgagctgccccaccccgcctttctgctgatcccccaggtgcagctgcagcagcctggcgccgagctggtgaagccaggcgccagcgtgaagctgtcctgcaaggccagcggctacaccttcaccggctactggatgcactgggtgaagcagagacccggccacggcctggaatggatcggcgagatcaaccccagcaacggccggaccaactacaacgagcggttcaagagcaaggccaccctgaccgtggacaagagcagcaccaccgccttcatgcagctgtccggcctgaccagcgaggacagcgccgtgtacttctgcgccagggactactacggcaccagctacaacttcgactactggggccagggcaccacactgaccgtgagcagcggcggagggggctctggcggcggaggatctgggggagggggcagcgacatccagatgacccagagcagcagcagcttcagcgtgagcctgggcgaccgggtgaccatcacctgtaaggccaacgaggacatcaacaaccggctggcctggtatcagcagacccccggcaacagccccaggctgctgatcagcggcgccaccaacctggtgaccggcgtgcccagccggtttagcggcagcggctccggcaaggactacaccctgaccatcacaagcctgcaggccgaggacttcgccacctactactgccagcagtactggtccacccccttcaccttcggcagcggcaccgagctggaaa

SEQ ID NO: 2 - protein, 229 aa, Artificial Sequence
  L (long) spacer L235D substitution
ESKYGPPCPPCPAPEFDGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - DNA, 673 bp, Artificial Sequence
  L (long) spacer L235D substitution
gaatctaagtacggaccgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 4 - protein, 28 aa, Artificial Sequence
  CD28tm
MFWVLVVVGGVLACYSLLVTVAFIIFWV

SEQ ID NO: 5 - DNA, 84 bp, Artificial Sequence
  CD28tm
atgttctgggtgctggtggtggtcggaggcgtgctggcctgctacagcctgctggtcaccgtggccttcatcatcttttgggtg

SEQ ID NO: 6 - protein, 41 aa, artificial sequence
  CD28 cytoplasmic
RSKRSRLLHSDYMNMTPRRPGPTRKHYQPYAPPRDFAAYRS

SEQ ID NO: 7 - DNA, 126 bp, Artificial Sequence
  41-BB
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 8 - DNA, 336 bp, Artificial Sequence
  CD3zeta
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 9 - DNA, 72 bp, Artificial Sequence
  T2A
ctcgagggcggcggagagggcagaggaagtcttctaacatgcggtgacgtggaggagaatcccggccctagg

SEQ ID NO: 10 - DNA, 1005 bp, Artificial Sequence
  EGFRt
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 11 - protein, 12 aa, Artificial Sequence
  S (small) spacer
ESKYGPPCPPCP

SEQ ID NO: 12 - DNA, 36 bp, Artificial Sequence
  S (small) spacer
gaatctaagtacggaccgccctgccccccttgccct

SEQ ID NO: 13 - protein, 119 aa, Artificial Sequence
  M (medium) spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - DNA, 357 bp, Artificial Sequence
  M (medium) spacer
gaatctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 15 - protein, 229 aa, Artificial Sequence
  L (long) spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - DNA, 687 bp, Artificial Sequence
  L (long) spacer
gaatctaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 17 - DNA, 123 bp, Artificial Sequence
  CD28 cytoplasmic (LL->GG, at 7th and 8th amino acid residues)
cggagcaagcggagcagaggcggccacagcgactacatgaacatgacccccagacggcctggccccacccggaagcactaccagccctacgccccacccagggactttgccgcctacagaagc

SEQ ID NO: 18 - protein, 41 aa, Artificial Sequence
  CD28 cytoplasmic (LL->GG, at 7th and 8th residues)
RSKRSRGGHSDYMNMTPRRPGPTRKHYQPYAPPRDFAAYRS

SEQ ID NO: 19 - protein, 229 aa, Artificial Sequence
  L (long) spacer L235D and N257Q substitutions (underlined)
ESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - DNA, 673 bp, Artificial Sequence
  L (long) spacer L235D and N257Q codon substitutions
gaatctaagtacggaccgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag